(19) United States
(12) Patent Application Publication
(10) Pub. No.: US 2025/0191746 A1
(43) Pub. Date: Jun. 12, 2025

(54) METHODS AND SYSTEMS FOR ENERGY SAVING CONTROL OF MEDICAL DEVICE

(71) Applicant: WUHAN UNITED IMAGING HEALTHCARE CO., LTD., Wuhan (CN)

(72) Inventor: Haichuan ZHANG, Wuhan (CN)

(73) Assignee: WUHAN UNITED IMAGING HEALTHCARE CO., LTD., Wuhan (CN)

(21) Appl. No.: 19/055,410

(22) Filed: Feb. 17, 2025

Related U.S. Application Data
(63) Continuation of application No. PCT/CN2023/081289, filed on Mar. 14, 2023.

(30) Foreign Application Priority Data
Sep. 8, 2022 (CN) 202211097238.1

(51) Int. Cl. G16H 40/40; G16H 40/63

(52) U.S. Cl. CPC G16H 40/40; G16H 40/63

(57) ABSTRACT

A method and system for energy saving control of a medical device are provided. The method for energy saving control includes obtaining first historical operation data for the medical device of a user; obtaining first predicted operation data by predicting, based on the first historical operation data, a user operation of the medical device, the first predicted operation data including a first predicted operation and a first predicted operation time corresponding to the first predicted operation; determining, based on the first predicted operation data and current operation data, an energy-saving strategy for controlling the medical device, the current operation data including a current operation and a current time; controlling the medical device to implement the energy-saving strategy.

### CROSS-REFERENCE TO RELATED APPLICATIONS

The present disclosure a continuation of International Application No. PCT/CN2023/081289, filed on Mar. 14, 2023, which claims Chinese Patent Application No. 202211097238.1, filed on Sep. 8, 2022, the entire contents of which are hereby incorporated by reference.

### TECHNICAL FIELD

The present disclosure relates to the field of computer technology, and in particular, to methods and systems for energy saving control of a medical device.

### BACKGROUND

With the advancement of modernization efforts in medical organizations, the installed volume of various medical devices (e.g., ultrasound diagnostic devices) is increasing, and the proportion of electricity consumed by these devices in the expenditure of medical organizations is rising. To reduce the electricity consumed by these medical devices, some medical devices are equipped with automatic energy-saving functions. For example, a waiting time is simply set, after which, if no operation is performed, the device enters a sleep mode. However, this manner has low energy-saving efficiency because it requires a fixed waiting time before entering the sleep mode. Moreover, in actual clinical scenarios, entering the sleep mode may not be anticipated by users. In such cases, the device needs to be awakened to continue to be used, and the time required to awaken the device is relatively long, which can affect the usage efficiency.

Therefore, it is desired to provide a method and system for energy saving control of a medical device that can effectively reduce the power consumption without affecting the usage efficiency of the medical device.

### SUMMARY

One of the embodiments of the present disclosure provides a method for energy saving control of a medical device. The method may include obtaining first historical operation data for the medical device of a user; obtaining a first predicted operation data by predicting, based on the first historical operation data, a user operation of the medical device, wherein the first predicted operation data includes a first predicted operation and a first predicted operation time corresponding to the first predicted operation; determining, based on the first predicted operation data and current operation data, an energy-saving strategy for controlling the medical device, wherein the current operation data includes a current operation and a current time; and controlling the medical device to implement the energy-saving strategy.

One of the embodiments of the present disclosure provides a system for energy saving control of a medical device. The system may include an acquisition module, a prediction module, a determination module, and a control module. The acquisition module may be configured to obtain first historical operation data for the medical device of a user. The prediction module may be configured to predict, based on the first historical operation data, a user operation of the medical device. The first predicted operation data may include a first predicted operation and a first predicted operation time corresponding to the first predicted operation. The determination module may be configured to determine, based on the first predicted operation data and current operation data, an energy-saving strategy for controlling the medical device. The current operation data may include a current operation and a current time. The control module may be configured to control the medical device to implement the energy-saving strategy.

One of the embodiments of the present disclosure provides a device for energy saving control of a medical device. The device may include at least one memory and at least one processor. The at least one memory may be configured to store computer instructions, and the at least one processor may be configured to execute at least some of the computer instructions to implement the method for energy saving control of any embodiment of the present disclosure.

### BRIEF DESCRIPTION OF THE DRAWINGS

The present disclosure is further illustrated in terms of exemplary embodiments. These exemplary embodiments are described in detail with reference to the drawings. These embodiments are non-limiting exemplary embodiments, in which like reference numerals represent similar structures, and wherein:

FIG. 1 is a schematic diagram illustrating a system for energy saving control according to some embodiments of the present disclosure;

FIG. 2 is a schematic diagram illustrating modules of a system for energy saving control shown according to some embodiments of the present disclosure;

FIG. 3 is a schematic diagram illustrating a structure of a device energy saving control according to some embodiments of the present disclosure;

FIG. 4 is a flowchart illustrating an exemplary method for energy saving control according to some embodiments of the present disclosure;

FIG. 5 is a flowchart illustrating an exemplary process of obtaining a trained prediction model according to some embodiments of the present disclosure;

FIG. 6 is a flowchart illustrating an exemplary process of determining whether to update a prediction model according to some embodiments of the present disclosure;

FIG. 7 is a flowchart illustrating an exemplary process of determining an energy-saving strategy according to some embodiments of the present disclosure;

FIG. 8 is a flowchart illustrating an exemplary process of determining an energy-saving strategy according to some embodiments of the present disclosure;

FIG. 9 is a flowchart illustrating an exemplary process of determining an energy-saving strategy according to some embodiments of the present disclosure;

FIG. 10 is a flowchart illustrating an exemplary process of determining saved energy consumption according to some embodiments of the present disclosure;

FIG. 11 is a schematic diagram illustrating a first predicted operation data according to some embodiments of the present disclosure;

FIG. 12 is a schematic diagram illustrating a first actual operation data according to some embodiments of the present disclosure; and

FIG. 13 is a schematic diagram illustrating an energy-saving management interface according to some embodiments of the present disclosure.

### DETAILED DESCRIPTION

To more clearly illustrate the technical solutions related to the embodiments of the present disclosure, a brief introduction of the drawings referred to the description of the embodiments is provided below. Obviously, the drawings described below are only some examples or embodiments of the present disclosure. Those having ordinary skills in the art, without further creative efforts, may apply the present disclosure to other similar scenarios according to these drawings. Unless obviously obtained from the context or the context illustrates otherwise, the same numeral in the drawings refers to the same structure or operation.

It should be understood that “system”, “device”, “unit” and/or “module” as used herein is a manner used to distinguish different components, elements, parts, sections, or assemblies at different levels. However, if other words serve the same purpose, the words may be replaced by other expressions.

As shown in the present disclosure and claims, the words “one”, “a”, “a kind” and/or “the” are not especially singular but may include the plural unless the context expressly suggests otherwise. In general, the terms “comprise”, “comprises”, “comprising”, “include”, “includes”, and/or “including”, merely prompt to include operations and elements that have been clearly identified, and these operations and elements do not constitute an exclusive listing. The methods or devices may also include other operations or elements.

The flowcharts used in the present disclosure illustrate operations that systems implement according to some embodiments of the present disclosure. It should be understood that the previous or subsequent operations may not be accurately implemented in order. Instead, each step may be processed in reverse order or simultaneously. Meanwhile, other operations may also be added to these processes, or a certain step or several steps may be removed from these processes.

In order to reduce the power consumed by a medical device, embodiments of the present disclosure provide a method and system for energy saving control of the medical device. Specifically, the method for energy saving control of the medical device may include obtaining first historical operation data for the medical device of a user; obtaining a first predicted operation data by predicting, based on the first historical operation data, a user operation of the medical device, the first predicted operation data including a first predicted operation and a first predicted operation time corresponding to the first predicted operation; determining, based on the first predicted operation data and current operation data, an energy-saving strategy for controlling the medical device, the current operation data including a current operation and a current time; and controlling the medical device to implement the energy-saving strategy. The method for energy saving control of the medical device disclosed in the embodiments of the present disclosure may be applied to a plurality of types of medical devices, provide a target energy-saving strategy for each of the medical devices by recognizing application scenarios of the medical devices, thereby reducing the power consumption of the medical device without affecting the usage efficiency.

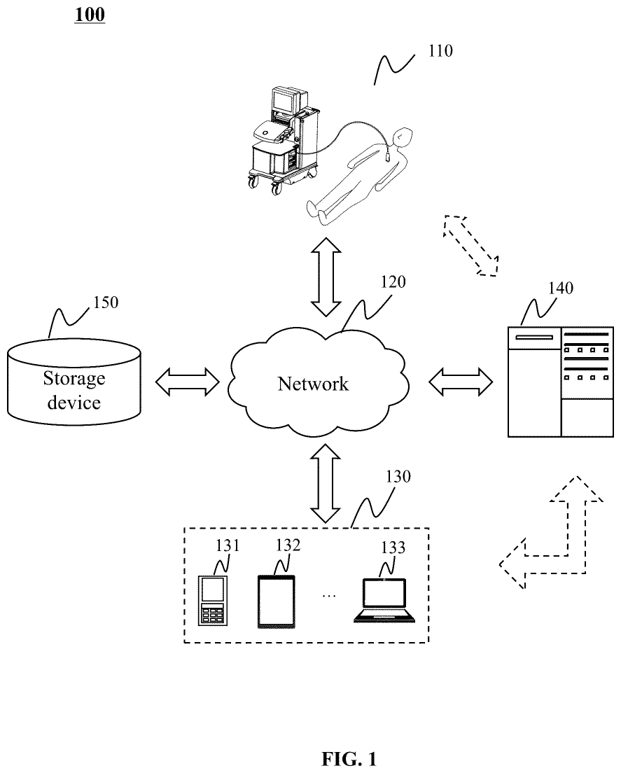

FIG. 1 is a schematic diagram illustrating a system for energy saving control according to some embodiments of the present disclosure.

As shown in FIG. 1, an system 100 for energy saving control may include a medical device 110, a network 120, a terminal 130, a processing device 140, and a storage device 150.

The medical device 110 may include an imaging device, an analyzing device, a therapeutic device, an assistive device, and other medical devices used for disease diagnosis or research purposes. In some embodiments, the medical device 110 may include an ultrasound device, and the ultrasound device may send higher frequency sound waves (e.g., ultrasound) to an object via a probe to perform an ultrasound scan. In some embodiments, the medical device 110 may include an ultrasound pulse echo imaging device, an ultrasound echo-Doppler imaging device, an ultrasound electronic endoscope, an ultrasound Doppler flow analysis device, an ultrasound human tissue measurement device, etc. In some embodiments, the object may include a biological object and/or a non-biological object. In some embodiments, the scanning modality of the medical device 110 may include an A-ultrasound, a B-ultrasound, an M-ultrasound, and/or a D-ultrasound, etc.

In some embodiments, the medical device 110 may also include an X-ray imaging device, a digital radiography (DR) device, a computed radiography (CR) device, a digital fluorography (DF) device, a biochemical immunoassay analyzer, a computed tomography (CT) device, a magnetic resonance (MR) device, a positron emission tomography (PET) imaging device, a digital subtraction angiography (DSA) device, an electrocardiogram device, a C-arm device, etc. The medical devices provided above are provided for illustrative purposes only and are not intended to limit the scope of the present disclosure.

In some embodiments, the medical device 110 may be disposed at a healthcare site or facility, such as a medical checkup center, a hospital room, a maternity ward, an examination room, an operating room, a rescue room, an ambulance, or the like. In some embodiments, the medical device 110 may be disposed at other locations, such as a marathon venue, an extreme sports venue, a racing venue, a disaster relief site, or the like. In some embodiments, the medical device 110 may also receive, via the network 120, control signals sent from the terminal 130 or the processing device 140 to perform an energy-saving strategy.

The network 120 may include any suitable network that contributes to the system 100 for energy saving control for exchanging information and/or data. In some embodiments, one or more other components of the system 100 for energy saving control (e.g., the medical device 110, the terminal 130, the processing device 140, the storage device 150, etc.) may exchange information and/or data with each other via the network 120. For example, the processing device 140 may obtain historical operation data (including first historical operation data, second historical operation data, or the like), category information of the medical device, or the like from the medical device 110 or the storage device 150 via the network 120. For example, the processing device 140 may obtain user instructions from the terminal 130 via the network 120 and determine whether to execute the energy-saving strategy based on the user instructions. The network 120 may be and/or may include a public network (e.g., the Internet), a private network (e.g., a local area network (LAN), a wide area network (WAN), etc.), a wired network (e.g., Ethernet), a wireless network (e.g., an 802.11 network, a Wi-Fi network, etc.), a cellular network (e.g., an LTE network), a frame relay network, a virtual private network (“VPN”), a satellite network, a telephone network, a router, a server computer, and/or a combination of one or more of these. For example, the network 120 may include one or a combination of one or more of a cable network, a wired network, a fiber optic network, a telecommunication network, a local area network (LAN), a wireless local area network (WLAN), a metropolitan area network (MAN), a public switched telephone network (PSTN), a Bluetooth™ network, a ZigBee™ network, a near-field communication network (NFC), or the like. In some embodiments, the network 120 may include one or more network access points. For example, the network 120 may include wired and/or wireless network access points, such as a base station and/or a network switching point, through which one or more of the components of the system 100 may be accessed by the system 100 to the network 120 for data and/or or information exchange.

In some embodiments, a user may operate the system 100 for energy saving control via the terminal 130. The terminal 130 may include a combination of one or more of a mobile device 131, a tablet 132, a laptop 133, etc. In some embodiments, an energy-saving strategy may be presented to the user via the terminal 130, which may receive the user instructions and transmit them to the processing device 140. In some embodiments, the mobile device 131 may include one or a combination of one or more of a smart home device, a wearable device, a mobile device, a virtual reality device, an augmented reality device, or the like. In some embodiments, the mobile device may include one or a combination of one or more of a cell phone, a personal digital assistant (PDA), a gaming device, a navigation device, a point-of-sale (POS) device, a laptop, a tablet, a desktop, or the like. In some embodiments, the virtual reality device and/or augmented reality device may include one or a combination of a virtual reality headset, virtual reality glasses, a virtual reality eyepiece, an augmented reality headset, augmented reality glasses, an augmented reality eyepiece, etc., and one or a combination of one or more of the same. For example, the virtual reality device and/or the augmented reality device may include Google Glass™, Oculus Rift™, Hololens™, Gear VR™, etc. In some embodiments, the terminal 130 may be part of the processing device 140. In some embodiments, the terminal 130 may be a part of the medical device 110.

The processing device 140 may process data and/or information obtained from the medical device 110, the terminal 130, and/or the storage device 150. For example, the processing device 140 may obtain first historical operation data from the medical device 110 or the storage device 150 and predict a user operation of the medical device based on the first historical operation data. In some embodiments, the processing device 140 may be a server or a cluster of servers. The server cluster may be centralized or distributed. In some embodiments, the processing device 140 may be local or remote. For example, the processing device 140 may access information and/or data stored at the medical device 110, the terminal 130, and/or the storage device 150 via the network 120. For example, the processing device 140 may be directly connected to the medical device 110, the terminal 130, and/or the storage device 150, thereby accessing information and/or data stored therein. In some embodiments, the processing device 140 may be executed on a cloud platform. For example, the cloud platform may include one or a combination of one or more of a private cloud, a public cloud, a hybrid cloud, a community cloud, a distributed cloud, an interconnected cloud, multiple clouds, or the like. In some embodiments, the processing device 140 may be performed by a computing device having one or more components. In some embodiments, the processing device 140 may be a part of the medical device 110 or the terminal 130.

The storage device 150 may store data, instructions, and/or other information. In some embodiments, the storage device 150 may store data obtained from the terminal 130 and/or the processing device 140. In some embodiments, the storage device 150 may store data and/or instructions executed or used by the processing device 140 to perform the exemplary methods described in the present disclosure. In some embodiments, the storage device 150 may include one or a combination of one or more of mass memory, removable memory, volatile read-write memory, read-only memory (ROM), or the like. Exemplary mass memory may include disks, optical disks, solid state drives, or the like. Exemplary removable memory may include flash drives, floppy disks, optical disks, memory cards, zipper disks, magnetic tapes, or the like. Exemplary volatile read-write memory may include random access memory (RAM). Exemplary RAM may include dynamic random access memory (DRAM), double data rate synchronized dynamic random access memory (DDR SDRAM), static random access memory (SRAM), thyristor random access memory (T-RAM), zero capacitance random access memory (Z-RAM), etc. Exemplary ROM may include mask read-only memory (MROM), programmable read-only memory (PROM), erasable programmable read-only memory (EPROM), electrically erasable programmable read-only memory (EEPROM), compact disc read-only memory (CD-ROM), and digital multi-purpose compact disc. In some embodiments, the storage device 150 may be implemented on a cloud platform. For example, the cloud platform may include one or a combination of one or more of a private cloud, a public cloud, a hybrid cloud, a community cloud, a distributed cloud, an interconnected cloud, multiple clouds, or the like.

In some embodiments, the storage device 150 may be connected to the network 120 to communicate with one or more other components in the system 100 for energy saving control (e.g., the processing device 140, the terminal 130, etc.). One or more components of the system 100 for energy saving control may access data or instructions stored in the storage device 150 via the network 120. In some embodiments, the storage device 150 may be directly connected to or in communication with one or more other components in the system 100 (e.g., the processing device 140, the terminal 130, etc.). In some embodiments, the storage device 150 may be a part of the processing device 140.

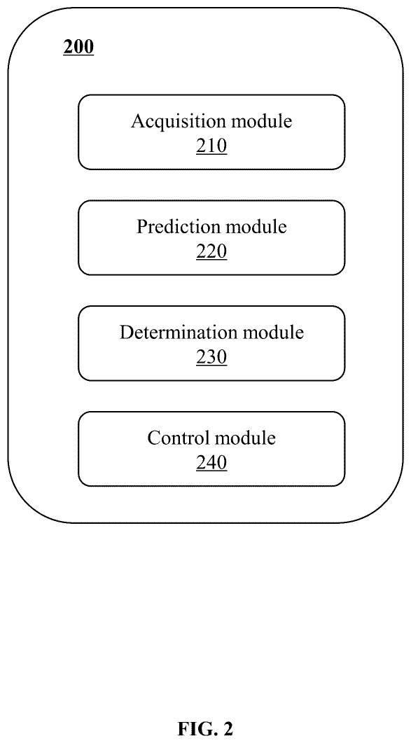

FIG. 2 is a schematic diagram illustrating modules of a system energy saving control shown according to some embodiments of the present disclosure.

As shown in FIG. 2, a system 200 for energy saving control may include an acquisition module 210, a prediction module 220, a determination module 230, and a control module 240. In some embodiments, the acquisition module 210, the prediction module 220, the determination module 230, and the control module 240 may be implemented by the processing device 140.

The acquisition module 210 may be configured to obtain first historical operation data for the medical device of a user. More descriptions regarding obtaining the first historical operation data may be found in the detailed description of operation 410, which will not be repeated here.

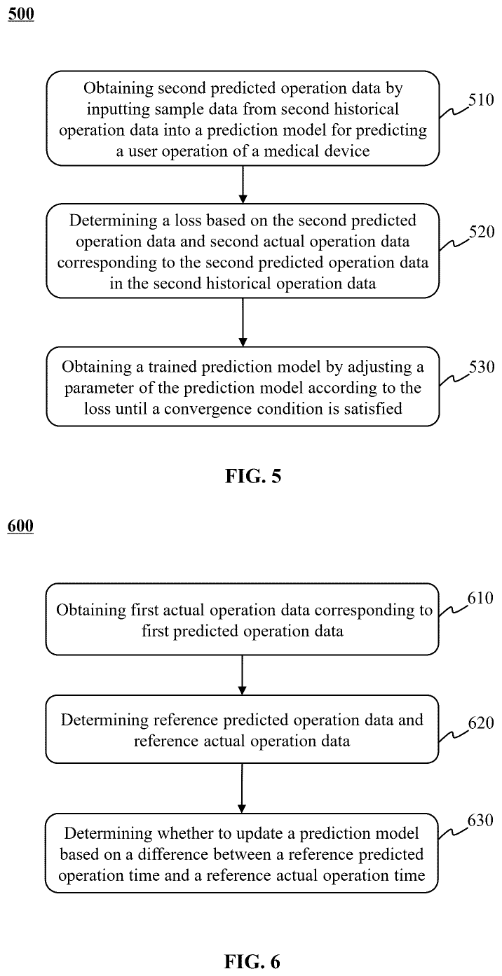

The prediction module 220 may be configured to obtain, based on the first historical operation data, the first predicted operation data by predicting a user operation of the medical device. The first predicted operation data may include a first predicted operation and a first predicted operation time corresponding to the first predicted operation. In some embodiments, the prediction module 220 may input the first historical operation data into a prediction model to obtain the first predicted operation data by predicting the user operation of the medical device. In some embodiments, the prediction module 220 may obtain the category information of the medical device and determine the prediction model corresponding to the category information based on the category information of the medical device. In some embodiments, the prediction module 220 may obtain first actual operation data corresponding to the first predicted operation data. The first actual operation data includes a first actual operation and a first actual operation time corresponding to the first actual operation and determine whether to update the prediction model based on the first predicted operation data and the first actual operation data. More descriptions regarding predicting the user operation of the medical device may be found in the detailed description of operation 420 and FIGS. 5-6, which will not be repeated here.

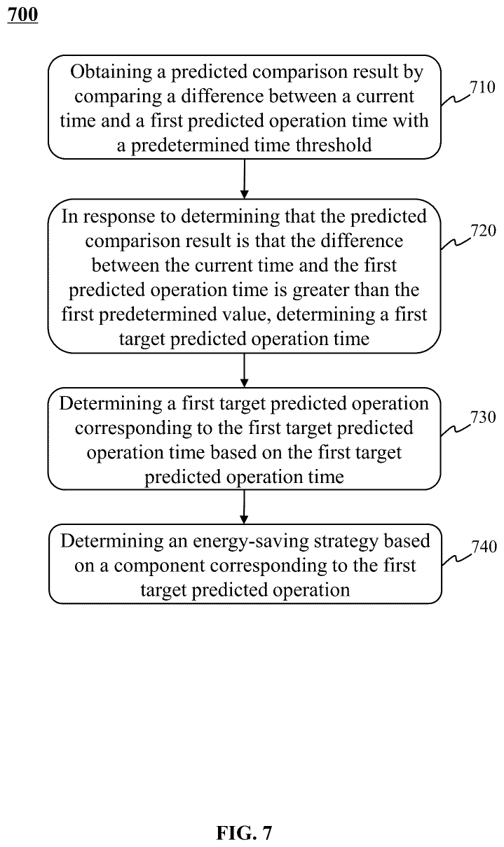

The determination module 230 may be configured to determine, based on the first predicted operation data and current operation data, an energy-saving strategy for controlling the medical device. The current operation data may include a current operation and a current time. In some embodiments, the determination module 230 may determine target predicted operation data of the user based on the first predicted operation data and the current operation data and determine the energy-saving strategy based on the target predicted operation data. In some embodiments, the determination module 230 may obtain a predicted comparison result by comparing, based on a predetermined time threshold, the first predicted operation data and the current operation data and determine the target predicted operation data of the user and/or a target energy-saving strategy based on the predicted comparison result. In some embodiments, the determination module 230 may obtain the predicted comparison result by comparing a difference between the current time and the first predicted operation time with the predetermined time threshold, and the predetermined time threshold may include a first predetermined value and a second predetermined value. More descriptions regarding determining the energy-saving strategy for controlling the medical device may be found in the detailed description of operation 430 and FIGS. 7-9, which will not be repeated here.

The control module 240 may be configured to control the medical device to perform the energy-saving strategy. More descriptions regarding controlling the medical device to perform the energy-saving strategy may be found in the detailed description of operation 440, which will not be repeated here.

It should be understood that the system and its modules shown in FIG. 2 may be implemented utilizing a variety of approaches. For example, in some embodiments the system and its modules may be implemented by hardware, software, or a combination of software and hardware.

It should be noted that the above description of the system and its modules is provided only for descriptive convenience, and does not limit the present disclosure to the scope of the cited embodiments. It is to be understood that for a person skilled in the art, after understanding the principle of the system, it may be possible to arbitrarily combine the individual modules or form a sub-system to be connected to the other modules without departing from this principle. For example, in some embodiments, e.g., the acquisition module 210, the prediction module 220, the determination module 230, and the control module 240 disclosed in FIG. 2 may be different modules in a single system, and also may be a single module that implements the functions of two or more of the above-described modules. For example, the individual modules may share a common storage module, and the individual modules may each have a respective storage module. Morphisms such as these are within the scope of protection of the present disclosure.

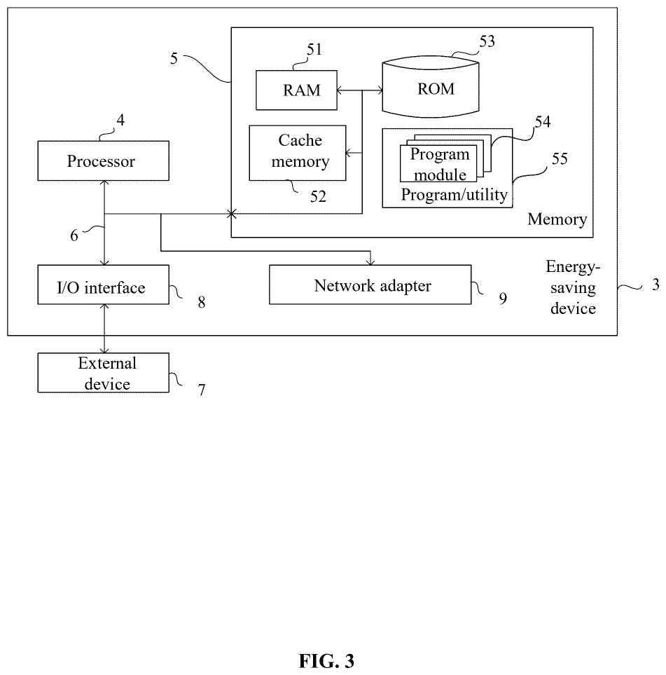

FIG. 3 is a schematic diagram illustrating a structure of a device 3 for energy saving control according to some embodiments of the present disclosure.

A device 3 for energy saving control may include at least one memory and at least one processor. The at least one memory is configured to store computer instructions, and the at least one processor is configured to execute at least some of the computer instructions to realize the method for energy saving control as described in any embodiment of the present disclosure.

In some embodiments, the medical device provided by embodiments of the present description may include an ultrasound device, an X-ray imaging device, a digital radiography device, a computed radiography device, a digital fluorography device, a biochemical immunoassay analyzer, a computed tomography device, a magnetic resonance device, a positron emission tomography imaging device, a digital subtraction angiography device, an electrocardiogram device, or the like. The medical devices provided above are provided for illustrative purposes only and are not intended to limit the scope of the present disclosure. In some embodiments, the device 3 for energy saving control may be performed by a computing device having one or more components. In some embodiments, the device 3 for energy saving control may be a part of the medical device or terminal. In some embodiments, the device 3 for energy saving control may be connected to a medical device to perform related functions.

In some embodiments, the components of the device 3 for energy saving control may include, but are not limited to, the at least one processor 4, the at least one memory 5, and a bus 6 connecting different system components, including the memory 5 and the processor 4. The bus 6 may include a data bus, an address bus, and a control bus.

The memory 5 may include volatile memory, such as random access memory (RAM) 51 and/or cache memory 52, and may further include read-only memory (ROM) 53. The memory 5 may also include a program/utility 55 having a set (at least one) of program modules 54. The program modules 54 may include, but are not limited to an operating system, one or more applications, and other program modules and program data. Each of these examples, or some combination thereof, may include an implementation of a network environment.

The processor 4 performs various functional applications and data processing, such as the method for energy saving control of the medical device described in any of the embodiments of the present disclosure, by running the computer instructions stored in the memory 5.

The device 3 for energy saving control may also communicate with one or more external devices 7 (e.g., keyboards, pointing devices, etc.). This communication may be realized through an input/output (I/O) interface 8. In addition, the device 3 for energy saving control may also communicate with one or more networks (e.g., a local area network (LAN), a wide area network (WAN), and/or a public network, such as the Internet) via a network adapter 9. As shown in FIG. 3, the network adapter 9 communicates with the other modules of the device 3 for energy saving control via the bus 6. It should be appreciated that, although not shown in FIG. 3, other hardware and/or software modules may be used in conjunction with the device 3 for energy saving control, including, but not limited to microcode, device drives, redundant processors, external disk drive arrays, RAID (disk array) systems, tape drives, data backup storage systems, etc.

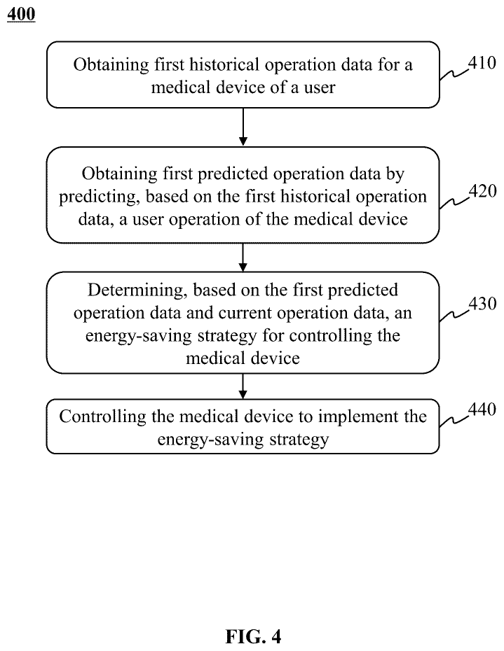

FIG. 4 is a flowchart illustrating an exemplary method for energy saving control according to some embodiments of the present disclosure.

The process 400 may be performed by a processing device (e.g., the processing device 140). For example, the process 400 may be implemented as an instruction set (e.g., an application program) that is stored in a memory internal or external to the system 100 for energy saving control. The processing device may execute the instruction set and, when executing the instructions, may be configured to execute process 400. The schematic diagram of the operation of process 400 presented below is illustrative. In some embodiments, the process may be accomplished by utilizing one or more additional operations that are not described and/or by omitting one or more operations discussed below. Additionally, the order of the operations of the process 400 illustrated in FIG. 4 and described below is not intended to be limiting.

In 410, first historical operation data for the medical device of a user is obtained. In some embodiments, operation 410 may be performed by the processing device 140 or the acquisition module 210.

The first historical operation data is the operation data for the medical device of the user before the current time. The operation data is data related to any operation performed on the medical device. The operation time corresponding to the operation data may include a start time and a stop time for a particular operation or may include only the start time. In some embodiments, the first historical operation data may be operation data at any time before the current time, such as the previous day or the previous week. The current time is typically the current system time of the medical device.

In some embodiments, the user is an operator or staff member of the medical device. In some embodiments, the user may be a healthcare staff member, e.g., a doctor, a nurse, etc.

In some embodiments, when a user operates the medical device at a historical moment, the processing device 140 may record the user operation data for each unit of the medical device. The historical moment is any moment before the current moment. In some embodiments, the unit of the medical device may include each of the components of the medical device. Merely by way of example, if the medical device is an ultrasound diagnostic device, the unit of the medical device may include a probe, a screen, a coupling agent heater, or the like. If the medical device is an X-ray imaging device, the unit of the medical device may include an emitter, a detector, or the like.

In some embodiments, the operation data may include actions entered by the user for each unit, e.g., the user turning the screen on again, the user turning off the heater, etc. Merely by way of example, in the case where the medical device is an ultrasound diagnostic device, the operation data may include operations such as powering on the device, heating the coupling agent, and entering an obstetric mode, as well as the operation time corresponding to the operation data.

In some embodiments, the operation data may also include data related to the automatic execution of certain operations within the medical device, and may include information about the entry of each unit into a certain state, e.g., entry into a preheating state or a working state, etc., and related energy consumption generated by the operation, etc.

In some embodiments, the processing device 140 may store and manage the recorded operation data according to time and units. Merely by way of example, the operation data of different units may be stored separately and the relevant operation data of this unit may be stored sequentially according to the chronological order. In some embodiments, the operation data may be stored in the storage device 150 or cloud storage, which may be accessed and managed by the processing device 140. In some embodiments, the management may include deleting records when the stored operation data exceeds a limited capacity. In some embodiments, the processing device 140 may delete a portion of the operation data for which the operation time is the earliest. In some embodiments, the management may further include encrypting the recorded operation data to control access, providing an access interface, synchronizing the operation data to the cloud for remote services, keeping the recorded operation data statistics, etc.

In some embodiments, since different users have different habits of operating the medical device, different accounts may be utilized to log in when different users operate the medical device to obtain the first historical operation data corresponding to the accounts. In some embodiments, when the medical device needs to turn on the smart energy-saving function, the processing device 140 may select the first historical operation data corresponding to the user from the stored operation data, specifically, the processing device 140 may select historical operation data within a certain time as the first historical operation data. In some embodiments, the user may also select the historical operation data within a certain time period on his or her own, e.g., the user may set an initial time and a cut-off time, and the processing device 140 may then select the historical operation data within that time period based on the initial time and cut-off time. In some embodiments, the processing device 140 may obtain the first historical operation data from the storage device 150 or cloud storage.

In 420, first predicted operation data is obtained by predicting, based on the first historical operation data, a user operation of the medical device. In some embodiments, operation 420 may be performed by the processing device 140 or the prediction module 220.

In some embodiments, the processing device 140 may predict the user operation based on a prediction algorithm or a prediction model, and the first predicted operation data includes a first predicted operation and a predicted operation time corresponding to the first predicted operation. The predicted operation time corresponding to the first predicted operation includes a start time and a stop time corresponding to the predicted operation or includes only the start time. The first predicted operation data may include at least one first predicted operation and a predicted operation time corresponding to the first predicted operation.

In some embodiments, the prediction algorithm may include a linear regression algorithm, a logistic regression algorithm, a gradient boosted decision tree algorithm (GBDT), a support vector machine algorithm, or the like. In some embodiments, the prediction model may be a trained machine learning model. In some embodiments, the machine learning model may include, but is not limited to, a neural network model, a convolutional neural network model, a visual geometric group network model, a full-resolution residual network model, a masked region convolutional neural network model, a multi-dimensional recurrent neural network model, and combinations of one or more of the same. In some embodiments, the machine learning model may be obtained by training based on a plurality of labeled historical operation data samples (e.g., second historical operation data), and the historical operation data samples include a historical operation and a historical operation time corresponding to the historical operation. The specific operations on how to train the prediction model may be found in the detailed description of FIG. 5, and will not be repeated here.

In some embodiments, the processing device 140 may input the acquired first historical operation data into a prediction model corresponding to the user or the user account, and the prediction model may output the corresponding first predicted operation data.

Since different users have different habits of using the medical device, even if the same medical device is used, the operation of the same medical device may be different due to the different departments in which the users (e.g., healthcare workers) are located. Additionally, even for the same examination, different users may have different habits and tendencies when using the medical device, such as whether it is used automatically, semi-automatically, or completely manually. Thus, based on the user account that the user logs into when using the medical device, the corresponding prediction model may be trained separately based on different classes or types of users.

In some embodiments, the processing device 140 may obtain category information of the medical device and determine a prediction model corresponding to the category information based on the category information of the medical device. The category information of the medical device may be information related to the user after categorizing the user. Merely by way of example, the user may be categorized based on the category of the medical device, e.g., the category of the medical device may include an ultrasound device, an X-ray imaging device, a CT device, and/or a magnetic resonance device, or the like. In some embodiments, the user may also be categorized based on departmental information, for example, the departmental information may include internal medicine, surgery, pediatrics, obstetrics and gynecology, oncology, or the like. In some embodiments, the processing device 140 may train different prediction models and store them based on the category information of the different medical devices, respectively, and simply select the corresponding prediction model when in use.

In some embodiments, the category information of the medical device may include category information of the medical device, and the processing device 140 may determine the prediction model corresponding to the category information based on the category information of the medical device. The category information of the medical device may include user information. In some embodiments, the processing device 140 may be trained for each user with a prediction model corresponding to the user account, and the prediction model corresponding to the user may be directly accessed when in use.

In some embodiments, the processing device 140 may also train a single prediction model based on a large amount of training data, which may accurately categorize the user directly based on the user account, and then correspondingly predict, based on categorization results, the operation data of the user. In this case, the processing device 140 may directly input the first historical operation data of the user into the prediction model, and the prediction model may automatically match and output the corresponding result without having to pre-train different types of a plurality of corresponding models. Relatively speaking, pre-training different types of prediction models yields more accurate prediction results than predicting all types of users through one single model.

In some embodiments, the prediction model may also be updated in real-time. Specifically, the processing device 140 may obtain first actual operation data corresponding to the first predicted operation data and determine, based on the first predicted operation data and the first actual operation data, whether to update the prediction model. Specific operations regarding determining whether to update the prediction model may be found in the relevant instructions of FIG. 6, and will not be repeated here.

In 430, based on the first predicted operation data and current operation data, an energy-saving strategy is determined for controlling the medical device. In some embodiments, operation 430 may be performed by the processing device 140 or the determination module 230.

The current operation data may include a current operation and a current time, the current operation may be a real operation for the medical device of the user at the current time, and the current operation data may be the data related to the user when performing the current operation. When the user performs an operation on the medical device, the processing device 140 may record in real-time the specific operation of the user and the corresponding operation time, and extract the current operation data to assist in determining the energy-saving strategy for the medical device.

In some embodiments, the processing device 140 may determine the energy-saving strategy for the medical device based on the first predicted operation data and the current operation data. The energy-saving strategy is the formulation of a strategy to be performed on the medical device that may reduce energy consumption to some extent.

In some embodiments, the processing device 140 may determine target predicted operation data for the user based on the first predicted operation data and the current operation data and determine the energy-saving strategy based on the target predicted operation data. In some embodiments, the processing device 140 may obtain a predicted comparison result by comparing, based on a predetermined time threshold, the first predicted operation data and the current operation data, and determine the target predicted operation data and/or target energy-saving strategy based on the predicted comparison result. In some embodiments, the processing device 140 may obtain the predicted comparison result by comparing a difference between the current time and the first predicted operation time with the predetermined time threshold. The predetermined time threshold may include a first predetermined value and a second predetermined value, or only one of the first predetermined value and the second predetermined value. The comparison of the difference between the current time and the first predicted operation time with the first predetermined value may be used to avoid disturbances caused by operation delays. When the difference is less than the first predetermined value where there may be operation delays, it is preferable not to perform the energy-saving operation. The comparison of the difference between the current time and the first predicted operation time with the first predetermined value may be used to determine whether the target component needs to be operated for a certain time period. When it does not need to be operated, then it is possible to execute the energy-saving operation. The specific operations regarding determining the energy-saving strategy may be found in the relevant descriptions of FIG. 7-FIG. 9 and will not be repeated here.

In some embodiments, the energy-saving strategy may include controlling the medical device to enter a sleep mode. The sleep mode may also be referred to as a low power mode, and entering the sleep mode may reduce energy consumption. Controlling the medical device to enter the sleep mode may power down some of the components in the controlling the medical device, or it may power down all the components in the controlling the medical device. In some embodiments, the energy-saving strategy may be determined to control the medical device to enter the sleep mode when the medical device does not continue to be operated for a certain amount of time after the medical device is stopped from being used.

In some embodiments, the energy-saving strategy may further include controlling a running component of the medical device to stop running. Controlling the component to stop running may be achieved by stopping the loading of a program associated with the component, and controlling the component to stop running may be achieved by controlling the component to power down.

In some embodiments, the energy-saving strategy may further include setting a predetermined time for the medical device, wherein the medical device may be automatically woken up when the predetermined time is reached.

In 440, the medical device is controlled to implement the energy-saving strategy. In some embodiments, operation 440 may be performed by the processing device 140 or the control module 240.

In some embodiments, the processing device 140 may control the medical device to manually execute the determined energy-saving strategy via a semi-automatic manner. For example, energy-savings may be made by manually controlling, or remotely controlling, relevant operations of the medical device. In some embodiments, the processing device 140 may also be automated in executing the energy-saving strategy, i.e., the processing device 140 sends a control command directly to the medical device to execute the energy-saving strategy, e.g., to enter a sleep mode or stopping the operation of a component. In some embodiments, the processing device 140 may also push the energy-saving strategy to the terminal to allow the user to select the corresponding energy-saving strategy and automatically execute the energy-saving strategy based on the user's selection.

In some embodiments, the processing device 140 may display, via a monitor or a terminal, an energy-saving management interface of the medical device. Merely by way of example, the management interface may include a generalized settings region and a statistics region. The general settings region may include a wait time for automatically turning off the screen, a device standby wait time, and an on/off button for the smart energy-saving feature. The statistical region may include a graph comparing the effect before and after energy-saving (as shown in FIG. 13) to visualize the effect of energy-saving by comparing the power consumed without turning on the energy-saving function (dashed portion) with the power actually consumed by the device (solid portion, through the method for energy saving control of the medical device provided by the embodiments of the present disclosure), and it may also show the value of the specific saved energy. Furthermore, the user may adjust the waiting time, the on or off of the smart energy-saving function, and the time range for the display of the effect comparison graph by using the relevant button.

In some embodiments, if there is a large error between the first predicted operation data and the current operation data and the error lasts for a certain period of time, it may be determined that the scenario in which the medical device is used has changed to a certain extent (a change of the location of the medical device, etc.). Specifically, the processing device 140 may compare the first predicted operation data with the current operation data. If the error between the first predicted operation data and the current operation data is large, the processing device 140 may stop executing the determined energy-saving strategy and update the prediction model to make a new prediction. The error between the first predicted operation data and the current operation data may include the difference between the current operation and the first predicted operation over a time period related to the time of the current operation, i.e., the type of operation, the count of operations, and other aspects of the combined error. Merely by way of example, when the processing device 140 predicts that the user will not use a relevant feature of the device (e.g., an image processing model) for 30 minutes and turns off the relevant component or software of the device, yet the user uses the feature a plurality of times within the 30-minute period, then the error exceeds a reasonable range. In some embodiments, if the error between the first predicted operation data and the current operation data is within the reasonable range, the energy-saving strategy may continue to be executed.

In summary, based on the prediction of the first historical operation data for the medical device of the user, the first predicted operation data is obtained. Under an actual use scenario corresponding to the current operation data, the energy-saving efficiency in the actual use scenario may be improved by controlling the medical device to execute the corresponding energy-saving strategy based on a relationship between the first predicted operation data and the current operation data.

It should be noted that the foregoing description of the process 400 is intended to be exemplary and illustrative only and does not limit the scope of application of the present disclosure. For a person skilled in the art, various corrections and changes may be made to the process 400 under the guidance of the present disclosure. However, these corrections and changes remain within the scope of the present disclosure.

FIG. 5 is a flowchart illustrating an exemplary process of obtaining a trained prediction model according to some embodiments of the present disclosure.

Process 500 may be performed by a processing device (e.g., the processing device 140). For example, the process 500 may be implemented as an instruction set (e.g., an application program) that is stored in a memory within or outside of the system 100 for energy saving control. The processing device may execute the instruction set and, when executing the instructions, may be configured to execute process 500. The schematic of the operation of process 500 presented below is illustrative. In some embodiments, the process may be accomplished by utilizing one or more additional operations that are not described and/or by omitting one or more operations discussed below. Additionally, the order of the operations of the process 500 illustrated in FIG. 5 and described below is not intended to be limiting. In some embodiments, process 500 may be used to realize operation 420 in process 400.

In 510, second predicted operation data is obtained by inputting sample data from the second historical operation data into the prediction model for predicting the user operation of the medical device. In some embodiments, operation 510 may be performed by the processing device 140 or the prediction module 220.

In some embodiments, the prediction model may be determined based on a second predicted operation data for the medical device of the user, the second predicted operation data may include a historical operation and a historical operation time corresponding to the historical operation. The historical operation corresponding to the historical operation time includes a start time and a stop time of the historical operation or includes only a start time. In some embodiments, the second historical operation data is data of a user operation of the medical device before the current time, which may be the same as or different from the first historical operation data. Merely by way of example, the second historical operation data may be obtained based on operation data other than the first historical operation data, and may also be obtained based on a portion of the first historical operation data and other historical operation data (other than the first historical data). For example, the second historical operation data may be the operation data of the previous week or the previous month. Usually, the amount of the second historical operation data is greater than the amount of the first historical operation data, and the greater the amount of the second historical operation data, the more accurate the results predicted by the prediction model.

In some embodiments, the training of the prediction model may be obtained based on a large amount of sample data of labeled second historical operation data. Specifically, a plurality of sample data of the second historical operation data with labels may be input into an initial prediction model. A loss may be calculated from a label and an output of the initial prediction model, and parameters of the prediction model may be adjusted based on a loss. Parameters of the initial prediction model may be randomly generated or obtained based on historical data. The training of the model is completed when predetermined conditions are satisfied, and the trained prediction model is obtained. In some embodiments, the output of the initial prediction model is the second predicted operation data, and the label is the actual operation corresponding to the second predicted operation data.

In some embodiments, the processing device 140 may input the sample data from the second historical operation data into the prediction model to predict the user operation of the medical device to obtain the second predicted operation data. The second predicted operation data may include a second predicted operation and a second predicted operation time corresponding to the second predicted operation. The second predicted operation time includes a start time and a stop time of the second predicted operation or includes only a start time.

In 520, a loss is determined based on the second predicted operation data and second actual operation data corresponding to the second predicted operation data in the second historical operation data. In some embodiments, operation 520 may be performed by the processing device 140 or the prediction module 220.

The second actual operation data may include a second actual operation and a second actual operation time corresponding to the second actual operation. The second actual operation is a real operation performed by the user for the medical device, the second actual operation corresponding to the second actual operation time includes a start time and a stop time of the second actual operation or includes only the start time. In some embodiments, the second actual operation time is later than a historical operation time in the sample data.

In some embodiments, the second actual operation data have a correspondence with the second predicted operation data. After obtaining the second predicted operation data, the generated actual operation data is the second actual operation data corresponding to the second predicted operation data. In some embodiments, the second predicted operation time and the second actual operation time may partially overlap or completely overlap.

In some embodiments, the processing device 140 determines an operational error based on the second predicted operation and the second actual operation. The operational error may represent, to some extent, a difference between the second predicted operation and the second actual operation. Specifically, the operational error may be obtained by coding each type of operation such as One-Hot coding and recording the operation with a coded value, and then calculating a mean square error (MSE) between the coded value corresponding to the second predicted operation and the coded value corresponding to the second actual operation.

In some embodiments, the processing device 140 may calculate a time error based on the second predicted operation time corresponding to the second predicted operation and the second actual operation time corresponding to the second actual operation. In some embodiments, the processing device 140 may obtain the time error by calculating a difference between the second predicted operation time and the second actual operation time.

In some embodiments, the processing device 140 may calculate the loss based on the operational error and the time error. Specifically, different weights may be set for the operational error and the time error depending on the actual situation, and the two may be weighted to obtain the loss.

In 530, a trained prediction model is obtained by adjusting a parameter of the prediction model according to the loss until a convergence condition is satisfied. In some embodiments, operation 530 may be performed by the processing device 140 or the prediction module 220.

In some embodiments, the processing device 140 may obtain the trained prediction model by iteratively updating the parameter of the prediction model based on the loss to satisfy the predetermined condition. The predetermined condition may be that the loss converges, or that the number of iterations reaches a threshold value, or the like.

In some embodiments, the prediction model may also be a fitting function, and the processing device 140 may be fitted to obtain the prediction model based on the second historical operation data. Specifically, the processing device 140 may be fitted to obtain the prediction model based on a polynomial fit, a nonlinear least squares fit, or the like. In some embodiments, when the degree of the fit of the prediction model obtained by fitting meets certain criteria, the fitting of the prediction model is completed for subsequent use.

In some embodiments, the processing device 140 may continually update the second predicted operation data to include the most recent operation data of the medical device by the user, and then optimize the trained prediction model based on the updated second predicted operation data. Specifically, a determination of whether to update the prediction model may be made based on the relevant description of FIG. 6.

In some embodiments, when the system for energy saving control is turned on or when the medical device is turned on or initialized with the energy-saving function, the processing device 140 may then obtain the second historical operation data to train the prediction model. Specifically, when the user uses the medical device, the processing device 140 may record relevant operation data of the medical device and train the prediction model based on the relevant operation data when the medical device is in an idle state. The prediction model consumes less network resources, may run in the background and does not interfere with the usage of the medical device. In some embodiments, the trained prediction model may be stored in the storage device 150 or cloud storage for ready access. In some embodiments, the prediction model may be encrypted to protect user privacy, and corresponding privacy protections may be set up for the user's operation data and access to the model. Merely by way of example, access may be provided by setting up a unified interface.

It should be noted that the foregoing description of the process 500 is intended to be exemplary and illustrative only and does not limit the scope of application of the present disclosure. For a person skilled in the art, various corrections and changes may be made to the process 500 under the guidance of the present disclosure. However, these corrections and changes remain within the scope of the present disclosure.

FIG. 6 is a flowchart illustrating an exemplary process of determining whether to update a prediction model according to some embodiments of the present disclosure.

Process 600 may be performed by a processing device (e.g., the processing device 140). For example, the process 600 may be implemented as an instruction set (e.g., an application program) that is stored in a memory within or external to the system 100 for energy saving control. The processing device may execute the instruction set and, when executing the instructions, may be configured to execute the process 600. The schematic of the operation of process 600 presented below is illustrative. In some embodiments, the process may be accomplished by utilizing one or more additional operations that are not described and/or by omitting one or more operations discussed below. Additionally, the order of the operations of process 600 illustrated in FIG. 6 and described below is not intended to be limiting. In some embodiments, the process 600 may be used to realize operation 420 of the process 400.

To ensure the accuracy of the prediction model, it is also determined whether to update the trained prediction model by following these operations.

In 610, first actual operation data corresponding to the first predicted operation data is obtained. In some embodiments, operation 610 may be performed by the processing device 140 or the prediction module 220.

The first actual operation data may include a first actual operation and a first actual operation time corresponding to the first actual operation. The first actual operation is a real operation for the medical device performed by a user at the first predicted operation time. The first actual operation data corresponds to the first predicted operation data. In some embodiments, the first predicted operation time and the first actual operation time may partially overlap or completely overlap. In some embodiments, the processing device 140 may record, in real time, data related to the operation of the medical device and obtain the first actual operation data from the medical device or the storage device.

In 620, reference predicted operation data and reference actual operation data are determined. In some embodiments, operation 620 may be performed by the processing device 140 or the prediction module 220.

In some embodiments, the reference predicted operation data may include a reference predicted operation and a reference predicted operation time corresponding to the reference predicted operation, and the reference actual operation data may include a reference actual operation and a reference actual operation time corresponding to the reference actual operation. In some embodiments, the reference predicted operation is any of the first predicted operations, and the reference actual operation is the same operation as the reference predicted operation in the first actual operation.

In some embodiments, the processing device 140 may randomly select any reference predicted operation data with corresponding reference actual operation data from the acquired first predicted operation data and first actual operation data. In some embodiments, the processing device 140 may select the reference predicted operation data with the corresponding reference actual operation data within a selected time period.

In 630, whether to update the prediction model is determined based on a difference between the reference predicted operation time and the reference actual operation time. In some embodiments, step 630 may be performed by the processing device 140 or the prediction module 220.

In some embodiments, the processing device 140 may compare the reference predicted operation data with corresponding reference actual operation data to determine whether to update the prediction model.

Specifically, the processing device 140 may determine whether to update the prediction model by comparing whether the difference between the reference predicted operation time and the reference actual operation time is greater than a fourth predetermined value. In some embodiments, if the difference between the reference predicted operation time and the reference actual operation time is greater than the fourth predetermined value, it may be determined that the prediction model needs to be updated. In some embodiments, if the difference between the reference predicted operation time and the reference actual operation time is not greater than the fourth predetermined value, it may be determined to maintain the prediction model unchanged.

In some embodiments, for any reference predicted operation, if the difference between the reference predicted operation time and the reference actual operation time (the reference actual operation and the reference predicted operation is the same operation) is greater than a fourth predetermined value, indicating that there is a large deviation between the predicted operation time and the actual operation time for a certain operation, at this time, it may be considered that the prediction model has a poor accuracy, and it is necessary to re-determine the prediction model to obtain accurate first predicted operation data, and execute a corresponding energy-saving strategy based on the accurate first predicted operation data to effectively realize energy-saving.

In some embodiments, for the at least two reference predicted operations, it is necessary to determine whether differences between at least two reference predicted operation times and the reference actual operation time are both greater than the fourth predetermined value to determine whether the prediction model needs to be updated. In some embodiments, for each of the at least two reference predicted operations, it is necessary to determine whether the difference between the reference predicted operation time and the reference actual operation time (the reference actual operation and the reference predicted operation are the same operation) is greater than the fourth predetermined value. In some embodiments, if differences between the at least two reference predicted operation times and the at least two reference actual operation times are both greater than the fourth predetermined value, it may be determined to update the prediction model. In some embodiments, if the differences between the at least two reference predicted operation times and the at least two reference actual operation times is not both greater than the fourth predetermined value, i.e., there exists at least one set of differences between the reference predicted operation times and the corresponding reference actual operation times less than or equal to the fourth threshold value, then it may be determined to maintain the prediction model unchanged. Merely by way of example, for three reference predicted operations, if differences between two of the three reference predicted operation times and the reference actual operation times are greater than the fourth predetermined value, but the differences between the other of the three reference predicted operation times and the reference actual operation times are not greater than the fourth predetermined value, it may be determined to maintain the prediction model unchanged.

In some embodiments, for at least two reference predicted operations, if there exists a difference between N sets of reference predicted operation times and corresponding reference actual operation times that is less than or equal to a fourth threshold value and M sets of reference predicted operation times and corresponding reference actual operation times is greater than the fourth threshold, and N is greater than or equal to M, it is indicated that most of the reference predicted operations predicted by the prediction model are still accurate, and it can be determined to maintain the prediction model unchanged. In some embodiments, if N is less than M, it is indicated that most of the reference predicted operations predicted by the prediction model are less accurate, and at this point, the prediction model may be considered to be re-determined.

In some embodiments, in order to avoid frequent re-determination of the prediction model, for at least two reference predicted operations, if the difference between the reference predicted operation time and the reference actual operation time (the reference actual operation and the reference predicted operation are the same operation) is greater than the fourth predetermined value, indicating that for both of the at least two operations, there is a large deviation between the predicted operation time and the actual operation time. At this time, the accuracy of the prediction model is considered to be poor, and it is necessary to re-determine the prediction model in order to obtain accurate first predicted operation data, and to execute a corresponding energy-saving strategy based on the accurate first predicted operation data, to effectively realize energy-saving.

It is to be noted that the smaller the fourth predetermined value is, the higher the accuracy requirement for the prediction model, which may be set according to the actual situation. In some embodiments, the fourth predetermined value may be set to 2 to 20 minutes, such as 5 minutes or 10 minutes, etc. The fourth predetermined value may be used to detect whether the prediction model is accurate or not. If the prediction result of the prediction model is inaccurate and the consecutive error is large, the model needs to be retrained.

In some embodiments, when it is determined that there is a need for updating the prediction model, the processing device 140 may re-determine the prediction model when the medical device is idle, such as by re-training the prediction model or re-fitting a function of the prediction model. The prediction model may be re-determined based on the third historical operation data, the third historical operation data may include the most recently generated actual operation data and may also include the first historical operation data and the second historical operation data.

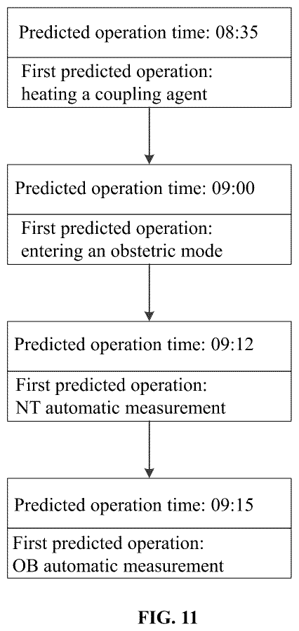

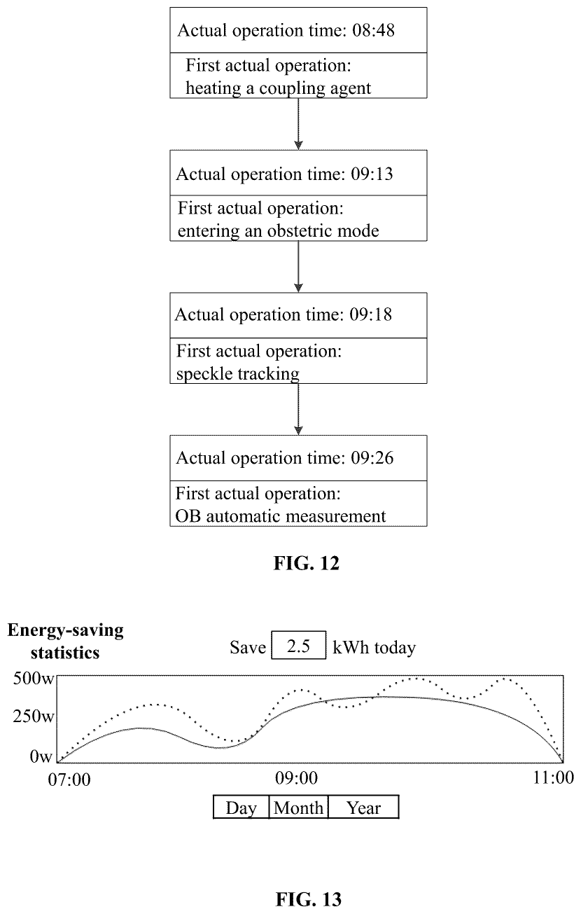

In some embodiments, if the medical device is an ultrasound diagnostic device, the fourth predetermined value is 10 minutes. As shown in FIGS. 11-12, the first actual operation may include heating the coupling agent, entering obstetrical mode, speckle tracking, and obstetric (OB) automatic measurement. The actual operation time corresponding to heating the coupling agent is 08:48, the actual operation time corresponding to entering obstetrical mode is 09:13, the actual operation time corresponding to speckle tracking is 09:18, and the actual operation time corresponding to OB automatic measurement is 09:26. Contrasting the first predicted operation data shown in FIG. 11 and the first actual operation data shown in FIG. 12, the same operations include heating the coupling agent, entering obstetrical mode, and OB automatic measurement, i.e., three reference predicted operations and three reference actual operations are included. For each of these three reference predicted operations, the difference between the reference predicted operation time and the reference actual operation time is greater than 10 minutes, specifically, the difference is 13 minutes, 13 minutes, and 11 minutes, respectively, and therefore prediction model needs to be re-determined.

It should be noted that the foregoing description of the process 600 is intended to be exemplary and illustrative only and does not limit the scope of application of the present disclosure. For a person skilled in the art, various corrections and changes may be made to the process 600 under the guidance of the present disclosure. However, these corrections and changes remain within the scope of the present disclosure.

FIG. 7 is a flowchart illustrating an exemplary process of determining an energy-saving strategy according to some embodiments of the present disclosure.

Process 700 may be performed by a processing device (e.g., the processing device 140). For example, the process 700 may be implemented as an instruction set (e.g., an application program) that is stored in a memory internal or external to the system 100 for energy saving control. The processing device may execute the instruction set and, when executing the instructions, may be configured to execute the process 700. The schematic diagram of the operation of process 700 presented below is illustrative. In some embodiments, the process may be implemented by utilizing one or more additional operations that are not described and/or by omitting one or more operations discussed below. Additionally, the order of the operations of process 700 illustrated in FIG. 7 and described below is not intended to be limiting. In some embodiments, process 700 may be used to implement operation 430 of process 400.

In 710, a predicted comparison result is obtained by comparing a difference between a current time and a first predicted operation time with a predetermined time threshold. In some embodiments, operation 710 may be performed by the processing device 140 or the determination module 230.

In some embodiments, the predetermined time threshold may include a first predetermined value, and the predicted comparison result is a relationship of the difference between the current time and the first predicted operation time with the predetermined time threshold. Specifically, the processing device 140 may compare the difference between the current time and the first predicted operation time with the first predetermined value. The processing device 140 may generally compare the difference between the current time and the corresponding start time of the predicted operation to the first predetermined value. In some embodiments, the processing device 140 may also compare the difference between the current time and the stop time corresponding to the predicted operation to the first predetermined value.

In 720, in response to determining that the predicted comparison result is that the difference between the current time and the first predicted operation time is greater than the first predetermined value, a first target predicted operation time is determined. In some embodiments, operation 720 may be performed by the processing device 140 or the prediction module 220.

In some embodiments, if the difference between the current time and the first predicted operation time is greater than a first predetermined value, the processing device 140 may determine the predicted operation time as the first target predicted operation time. In some embodiments, the first predetermined value may be set according to the actual situation or the results of the experiment, and the first predetermined value may be within a range of 5-30 minutes. In some embodiments, the first predetermined value may be within a range of 10-30 minutes. For example, the first predetermined value may be set to be within a range of 20 minutes-30 minutes, etc.

In some embodiments, a comparison of the difference between the current time and the first predicted operation time with the predetermined threshold may be used to determine an error. Specifically, if the prediction comparison result is the difference greater than the first predetermined value, interference may be avoided to a certain extent. Usually, the user may not be in full accordance with the predicted results when using the medical device, and there may be some variations. If it is predicted that the user is going to carry out a certain operation after 3 minutes, but actually the user may not be able to carry out the operation until 5 minutes later. Then, if a component is turned off after 3 minutes, it may affect the actual user experience. Therefore, the difference between the predicted operation time and the current time should be greater than a threshold value to avoid errors caused by operational delays affecting the user, so that the user will have a better experience.

In 730, a first target predicted operation corresponding to the first target predicted operation time is determined based on the first target predicted operation time. In some embodiments, operation 730 may be performed by the processing device 140 or the prediction module 220.

In some embodiments, since the first target predicted operation time is the predicted operation time, the predicted operation may be determined as the corresponding first target predicted operation.

In 740, the energy-saving strategy is determined based on a component corresponding to the first target predicted operation. In some embodiments, operation 740 may be performed by the processing device 140 or the prediction module 220.

In some embodiments, the processing device 140 may determine whether the first target component corresponding to the first target predicted operation is operating. If the first target component is operating and the first target component is not the component corresponding to the target operation, the energy-saving strategy may be determined to control the first target component to stop operating. The first target component is an operating component corresponding to the first target predicted operation. The target operation includes a current operation and a first predicted operation. A difference between the predicted operation time of the first predicted operation and the current time is less than or equal to the first predetermined value. As the difference between the predicted operation time and the current time is greater than the first predetermined value, interference caused by delayed operation may be excluded, and at this time, if the component corresponding to the first target component is not a component corresponding to the target operation, it may be indicated that the predicted operation is not performed, thus the first target component performing the predicted operation need not be in an operating state. In some embodiments, the processing device 140 may further control the first target component to stop operating according to the energy-saving strategy to achieve energy-saving.

In some embodiments, if the first target component corresponding to the first target predicted operation is operating and the first target component is the component corresponding to the target operation, the first target component may be controlled to continue to operate so that the current operation may be completed normally, and the process of the method for energy saving control may be stopped.

In some embodiments, the first predicted operation data outputted by the prediction model may include a single first predicted operation or may include a plurality of first predicted operations. Similarly, the first target predicted operation may include a single first predicted operation or may include a plurality of first predicted operations. The first target components corresponding to the first target predicted operation may be one or two and may also be a plurality of first target components.

In some embodiments, after controlling the first target component to stop operating, the processing device 140 may control the first target component to operate again in response to an actual operation of the user against the medical device.

Merely by way of example, if the medical device is an ultrasound diagnostic device, the first predicted operation includes speckle tracking, OB automatic measurement, and nuchal translucency (NT) automatic measurement. The predicted operation time corresponding to speckle tracking is 9:00, the time predicted operation time corresponding to OB automatic measurement is 9:30, the predicted operation time corresponding to NT automatic measurement is 9:40, and the first predetermined value is 30 minutes. The predicted operation times at which the difference with the current time is greater than 30 minutes, i.e., 9:30 and 9:40, are determined as the first target predicted operation time. The first target predicted operation corresponding to the first target predicted operation time includes an OB automatic measurement and an NT automatic measurement, and the target component corresponding to the first target predicted operation includes a probe, a graphics processing unit (GPU), and a coupling agent heater. It is assumed that the current operation is heating the coupling agent, the corresponding component is the coupling agent heater, and the first target component in operation is the GPU. The target operation includes the current operation and a first predicted operation (i.e., speckle tracking) corresponding to a predicted operation time (i.e., 9:00) that has a difference with the current time, i.e., 8:40, less than or equal to 30 minutes, and it is determined that the GPU is not a component corresponding to the target operation. At this time, the energy-saving strategy may be determined as controlling the GPU to stop operating, thereby realizing energy-saving.

In some embodiments, the processing device 140 may record each operation of the medical device in real-time, the operation may include an operation of the medical device by the user, or an operation during the operation of the device. The processing device 140 may obtain the situation of the first target component from the processor of the medical device, which may be obtained by other means. For example, a camera may additionally be disposed outside the medical device to obtain real-time the situation of the operation of various components in the medical device.

It should be noted that the foregoing description of the process 700 is intended to be exemplary and illustrative only and does not limit the scope of application of the present disclosure. For a person skilled in the art, various corrections and changes may be made to the process 700 under the guidance of the present disclosure. However, these corrections and changes remain within the scope of the present disclosure.

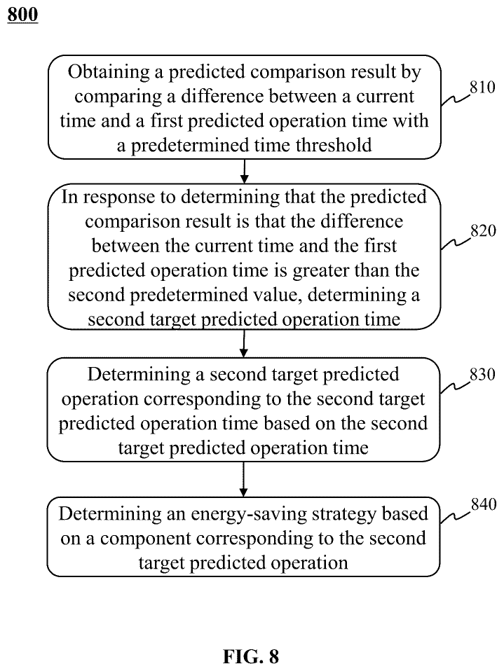

FIG. 8 is a flowchart illustrating an exemplary process of determining an energy-saving strategy according to some embodiments of the present disclosure.

Process 800 may be performed by a processing device (e.g., the processing device 140). For example, the process 800 may be implemented as an instruction set (e.g., an application program) that is stored in a memory within or outside of the system 100 for energy saving control. The processing device may execute the instruction set and, when executing the instructions, may be configured to execute the process 800. The schematic of the operation of process 800 presented below is illustrative. In some embodiments, the process may be accomplished by utilizing one or more additional operations that are not described and/or by omitting one or more operations discussed below. Additionally, the order of the operations of process 800 illustrated in FIG. 8 and described below is not intended to be limiting. In some embodiments, process 800 may be used to implement operation 430 of process 400.

In 810, a predicted comparison result is obtained by comparing a difference between a current time and a first predicted operation time with a predetermined time threshold. In some embodiments, operation 810 may be performed by the processing device 140 or the determination module 230.

In some embodiments, the predetermined time threshold may include a second predetermined value, and the predicted comparison result is a relationship of the difference between the current time and the first predicted operation time with the second predetermined value. Specifically, the processing device 140 may compare the difference between the current time and the first predicted operation time with the second predetermined value. The processing device 140 may generally compare the difference between the current time and the corresponding start time of the predicted operation to the second predetermined value. In some embodiments, the processing device 140 may also compare the difference between the current time and the stop time corresponding to the predicted operation to the second predetermined value.

In 820, in response to determining that the predicted comparison result is that the difference between the current time and the first predicted operation time is smaller than the second predetermined value, a second target predicted operation time is determined. In some embodiments, operation 820 may be performed by the processing device 140 or the prediction module 220.

In some embodiments, if the difference between the current time and the first predicted operation time is less than the second predetermined value, the processing device 140 may determine the predicted operation time as a second target predicted operation time. In some embodiments, the second predetermined value may be set according to the actual situation or experimental results, and the second predetermined value may be within a range of 5-60 minutes, for example, it may be set to 20 minutes, 30 minutes, or 40 minutes, etc.

In some embodiments, the first predicted operation data may include a single first predicted operation or a plurality of first predicted operations, and the number of predicted operations may be preset. Merely by way of example, the medical device is an ultrasonic diagnostic device for example, and the first predicted operation includes heating the coupling agent, entering obstetrical mode, NT automatic measurement, and OB automatic measurement. During several successive operations, the user may stop operating the medical device for a time period.

In 830, a second target predicted operation corresponding to the second target predicted operation time is determined based on the second target predicted operation time. In some embodiments, operation 830 may be performed by the processing device 140 or the prediction module 220.

In some embodiments, since the second target predicted operation time is the predicted operation time, the predicted operation may be determined as the second target predicted operation corresponding to the second target predicted operation time.

In 840, the energy-saving strategy is determined based on a component corresponding to the second target predicted operation. In some embodiments, operation 840 may be performed by the processing device 140 or the prediction module 220.

In some embodiments, the processing device 140 may determine whether neither the component corresponding to the second target predicted operation nor a component corresponding to the current operation includes a second target component that is currently operating. If so, the energy-saving strategy may be determined to control the second target component to stop operating. In some embodiments, after controlling the second target component to stop operating, the second target component may be controlled to operate again in response to an actual operation of the user against the medical device.

In some embodiments, starting from the current time, if it is predicted that the second target component may not be used during the subsequent time period within the second predetermined value, the second target component is controlled to stop operating, which may effectively achieve energy savings.

In some embodiments, the first predicted operation data output by the prediction model may include a single first predicted operation or may include a plurality of first predicted operations. The second target component is a component that is currently operating, and the number of second target component may be one, two, or more.

Merely by way of example, if the medical device is an ultrasound diagnostic device, as shown in FIGS. 11-12, the first predicted operation includes heating the coupling agent, entering the obstetrical mode, NT automatic measurement, and OB automatic measurement. The predicted operation time corresponding to the time of heating the coupling agent is 08:35, the predicted operation time corresponding to entering obstetrical mode is 09:00, the predicted operation time corresponding to NT automatic measurement is 09:12, the predicted operation time corresponding to OB automatic measurement is 09:15, the current time is 8:30, and the second predetermined value is 40 minutes. The predicted operation time in which the difference with the current time is less than 40 minutes is determined to include 08:35 and 09:00, i.e., the second target predicted operation time includes 08:35 and 09:00. The second target predicted operation time corresponding to the second target predicted operation time includes heating the coupling agent and entering the obstetrical mode, and the component corresponding to the second target predicted operation includes a coupling agent heater and a probe. Assuming that the current operation is heating the coupling agent, the component corresponding to the current operation is a coupling agent heater, and the second target component that is currently in operation includes a coupling agent heater and a GPU. It is determined that neither the components corresponding to the second target predicted operation nor the components corresponding to the current operation include a GPU that is currently running. At this time, it may be determined that the energy-saving strategy is that controlling the GPU to stop operating may realize energy-saving.

It should be noted that the foregoing description of the process 800 is intended to be exemplary and illustrative only and does not limit the scope of application of the present disclosure. For a person skilled in the art, various corrections and changes may be made to the process 800 under the guidance of the present disclosure. However, these corrections and changes remain within the scope of the present disclosure.

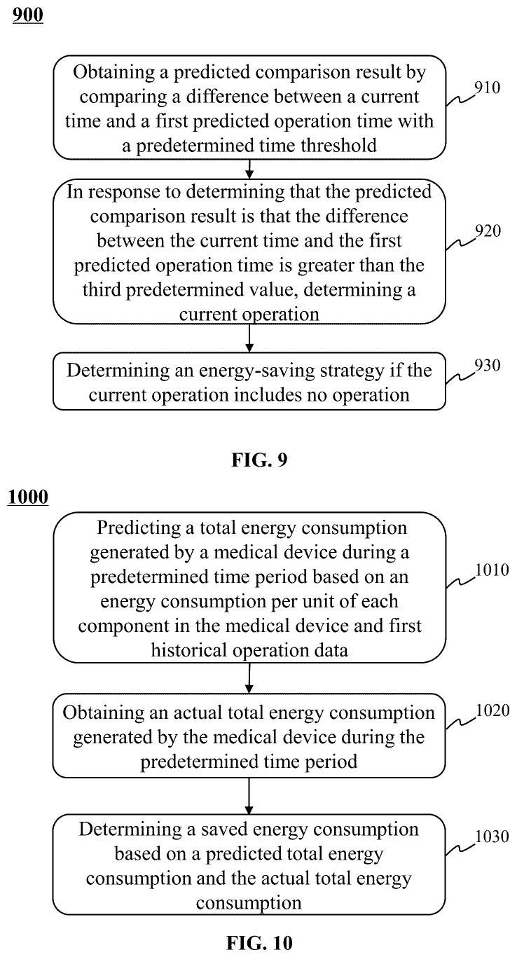

FIG. 9 is a flowchart illustrating an exemplary process of determining an energy-saving strategy according to some embodiments of the present disclosure.

Process 900 may be performed by a processing device (e.g., the processing device 140). For example, the process 900 may be implemented as an instruction set (e.g., an application program) that is stored in a memory within or outside of the system 100 for energy saving control. The processing device may execute the instruction set and, when executing the instructions, may be configured to execute the process 900. The schematic diagram of the operation of process 900 presented below is illustrative. In some embodiments, the process may be accomplished by utilizing one or more additional operations that are not described and/or by omitting one or more operations discussed below. Additionally, the order of the operations of process 900 illustrated in FIG. 9 and described below is not intended to be limiting. In some embodiments, process 900 may be used to implement operation 430 of process 400.

In 910, a predicted comparison result is obtained by comparing a difference between a current time and a first predicted operation time with a predetermined time threshold. In some embodiments, operation 910 may be performed by the processing device 140 or the determination module 230.

In some embodiments, the predetermined time threshold may include a third predetermined value, and the predicted comparison result is a relationship of the difference between the current time and the first predicted operation time with the third predetermined value. Specifically, the processing device 140 may compare the difference between the current time and the first predicted operation time with the third predetermined value. The processing device 140 may generally compare the difference between the current time and the corresponding start time of the predicted operation to the third predetermined value. In some embodiments, the processing device 140 may also compare the difference between the current time and the stop time corresponding to the predicted operation to the third predetermined value.

In 920, in response to determining that the predicted comparison result is that the difference between the current time and the first predicted operation time is greater than the third predetermined value, a current operation is determined. In some embodiments, operation 920 may be performed by the processing device 140 or the prediction module 220.

In some embodiments, the third predetermined value may be set according to the actual situation or the experimental results, and the third predetermined value may be within a range of 30 minutes to 2 hours, for example, the third predetermined value may be set to 1 hour, or the like.

In 930, if the current operation includes no operation, an energy-saving strategy is determined. In some embodiments, operation 930 may be performed by the processing device 140 or the prediction module 220.

In some embodiments, if the difference between the time of a predicted operation closest to the current time in the first predicted operation data and the current time is greater than the third predetermined value, and there is no current operation, the energy-saving strategy may be determined as controlling the medical device to enter a sleep mode. At this time, since there is no predicted operation for the next time period, and there is no current operation, entering the sleep mode may effectively save power consumption of the device.

In some embodiments, if there is no actual operation at present and an operation is predicted to occur after a time period from the third predetermined value of the current time, the medical device may be controlled to enter the sleep mode without waiting for a time period to enter the sleep mode, which may effectively improve the energy-saving efficiency. At the same time, since no operation is predicted to occur within the time period from the third predetermined value of the current time based on historical operation data, entering the sleep mode at this time is in line with the user's expectations, which may enhance the user experience.

In some embodiments, if the difference between the time of the predicted operation closest to the current time in the first predicted operation data and the current time exceeds a third predetermined value, and there is no current operation, it is also possible to control all the components that are currently operating to stop operating to realize energy-saving.

In some embodiments, the predetermined time threshold may include one or more of a first predetermined value, a second predetermined value, or a third predetermined value. For example, the predetermined time threshold may include only the second predetermined value. As another example, the predetermined time threshold may include all of the first predetermined value, the second predetermined value, and the third predetermined value.

In some embodiments, the processing device 140 may simultaneously compare the difference between the current time and the predicted operation time to at least two of three predetermined time thresholds (e.g., the first predetermined value, the second predetermined value, and the third predetermined value). For example, the difference between the current time and the predicted operation time is compared with both of the first predetermined value and the second predetermined value, the difference between the current time and the predicted operation time is compared with both of the first predetermined value and the third predetermined value, the difference between the current time and the predicted operation time is compared with both of the first predetermined value and the third predetermined value, and the difference between the current time and the predicted operation time is compared with all of the first predetermined value, the second predetermined value, and the third predetermined value. With the setting of the first predetermined value, the second predetermined value, and the third predetermined value, energy-saving may be realized with reduced user perception, unnecessary business interruptions may be reduced, and user experience may be improved. In some embodiments, the difference between the current time and the first predicted operation time may simultaneously satisfy a relationship with a plurality of predetermined values. At this time, it is only necessary to compare the difference with the plurality of predetermined values separately, and further determine the corresponding energy-saving strategies accordingly. For the same first predicted operation data and the current operation data, the process 700, the process 800, and the process 900 may be executed simultaneously.

It is to be noted that when it is determined that the plurality of energy-saving strategies are simultaneously satisfied based on the first predicted operation data and current operation data, a corresponding priority may be set for different energy-saving strategies according to the actual situation, and then according to the order of the priorities, the corresponding energy-saving strategies are executed. Specifically, for the same component, if the determined energy-saving strategies are contradictory, the priority may be determined based on the determined energy-saving operation and the way in which the energy-saving strategies are determined. In some embodiments, based on the type of energy-saving operation, if the determined energy-saving operation for the same component in the determined energy-saving strategy includes stopping operation, sleeping, and no operation, the no operation, i.e., remaining the component on, may be prioritized. In some embodiments, if the contradictory energy-saving strategies are determined by different predetermined time thresholds, the energy-saving strategy determined by one of the first predetermined value, the second predetermined value, and the third predetermined value may be prioritized as an energy-saving strategy. In some embodiments, if the plurality of determined energy-saving strategies are not contradictory, the corresponding energy-saving strategies may be executed separately. In general, it is also possible to analyze the situation according to the specific circumstances, to determine the most appropriate energy-saving strategy, which achieves the purpose of energy-saving without affecting the user's experience.

It should be noted that the foregoing description of the process 900 is intended to be exemplary and illustrative only and does not limit the scope of application of the present disclosure. For a person skilled in the art, various corrections and changes may be made to the process 900 under the guidance of the present disclosure. However, these corrections and changes remain within the scope of the present disclosure.

FIG. 10 is a flowchart illustrating an exemplary process of determining saved energy consumption according to some embodiments of the present disclosure.

Process 1000 may be performed by a processing device (e.g., the processing device 140). For example, the process 1000 may be implemented as an instruction set (e.g., an application program) that is stored in a memory internal or external to the system 100 for energy saving control. The processing device may execute the instruction set and, when executing the instructions, may be configured to execute the process 1000. The schematic diagram of the operation of process 1000 presented below is illustrative. In some embodiments, the process may be accomplished by utilizing one or more additional operations that are not described and/or by omitting one or more operations discussed below. Additionally, the order of the operations of process 1000 illustrated in FIG. 10 and described below is not intended to be limiting.

In some embodiments, an energy consumption saved from implementing the method for energy saving control described in some embodiments of the present disclosure may be determined by the following operations.

In 1010, a total energy consumption generated by the medical device during a predetermined time period is predicted based on a unit energy consumption of each component in the medical device and the first historical operation data.

In some embodiments, the predetermined time period may be set according to the actual situation, for example, it may be set to 1 day, 5 days, 1 week, 10 days, etc.

Specifically, the total energy consumption generated by the medical device during the predetermined time period predicted based on the first historical operation data refers to the total energy consumption that would have been generated by not implementing the energy-saving strategies of some embodiments of the present disclosure. In some embodiments, a corresponding component may be determined based on a historical operation in the first historical operation data, and a starting operation time of each component may be determined based on a historical operation time corresponding to the historical operation. Without execution of the energy-saving strategy, it is considered that the components do not stop after they start operating until the medical device is shut down. Based on the shutdown time of the medical device and the starting operation time of each component, the operation duration of each component may be predicted, and finally, based on the unit energy consumption of each component and the operation duration of each component, the total energy consumption generated by all the components may be calculated, which may be used as the total energy consumption generated by the medical device.

In some embodiments, the first historical operation data may be related to the predetermined time period, or the first historical operation data may not be related to the predetermined time period. For example, the first historical operation data may be operation data within any historical time period, and the predetermined time period may be of the same or a different length of time than the historical time period corresponding to the first historical operation data. In some embodiments, to make the prediction results as accurate as possible, the historical time period corresponding to the first historical operation data may be as close as possible to the predetermined time period. For example, the historical time period may be the most recent time period before the predetermined time period, which may be the previous day, the previous week, the previous two weeks, the previous month, etc.

In some embodiments, the first historical operation data may be the operation data of the previous day, and the predetermined time period may be a day. The total energy consumption generated by the medical device in the previous day is calculated based on the operation duration of each component in the previous day and the energy consumption per unit of each component, and the total energy consumption is designated as a predicted total energy consumption generated by the medical device in one day.

In some embodiments, the first historical operation data may be the operation data of the previous week, and the predetermined time period may be one day. The total energy consumption generated by the medical device for each day of the previous week is calculated based on the operation duration of each component and the unit energy consumption of each component for each day of the previous week, a weighted sum is performed to obtain a predicted total energy consumption generated by the medical device in a day. The weights of the total energy consumption generated by the medical device for each day of the previous week may be set according to the actual situation. Merely by way of example, the weights of the total energy consumption generated by the medical device for each day of the preceding week may all be set to 1/7, i.e., the total energy consumption generated by the medical device in the preceding week is averaged to obtain the predicted total energy consumption generated by the medical device in a day.

In 1020, an actual total energy consumption generated by the medical device during the predetermined time period is obtained.

The total energy consumption actually generated by the medical device during the predetermined time period is the total energy consumption generated by implementing the energy-saving strategy.

In 1030, the saved energy consumption is determined based on the predicted total energy consumption and the actual total energy consumption.

In some embodiments, subtracting the predicted total energy consumption from the actual total energy consumption may yield the saved energy consumption after employing the energy-saving strategy. To facilitate the user to view the saved energy consumption by the medical device, the saved energy consumption by the medical device during the predetermined time period may also be displayed in a display interface or terminal of the medical device.

In the example shown in FIG. 13, the dashed line reflects the power consumed by an ultrasound diagnostic device on a certain day without adopting the method for energy saving control provided in the present embodiment, and the solid line reflects the power consumed by the ultrasound diagnostic device on a certain day adopting the method for energy saving control provided in the present embodiment. As can be seen from FIG. 13, 2.5 kWh of energy may be saved on a certain day by adopting the method for energy saving control provided by the embodiments of the present disclosure. By displaying device power consumption-time statistics, the actual power consumption after energy-saving and the expected increase in power consumption without adopting the energy-saving function may be highlighted to turn on or turn off the energy-saving function and present the energy-saving optimization effect through a visual and interactive module, providing the user with visual comparisons.

It should be noted that the foregoing description of the process 1000 is intended to be exemplary and illustrative only and does not limit the scope of application of the present disclosure. For a person skilled in the art, various corrections and changes may be made to the process 1000 under the guidance of the present disclosure. However, these corrections and changes remain within the scope of the present disclosure.

The embodiments of the present disclosure may include but are not limited to the following beneficial effects. First, the actual clinical scenarios, where the medical device is utilized, are identified, and a target energy-saving strategy for each medical treatment is provided to reduce the power consumption of the medical device without affecting the usage efficiency. Second, the accuracy of the prediction of operations is improved by training the prediction model according to the different categories or types of users respectively. Third, energy-saving may be achieved with reduced user perception, reducing unnecessary business interruptions, and improving user experience. Fourth, a visual and interactive module may be used to turn on or turn off the energy-saving function and present the energy-saving optimization effect, providing users with visual comparisons.

It should be noted that beneficial effects that may be produced by different embodiments are different, and the beneficial effects that may be produced in different embodiments may be any one or a combination of any of the foregoing, or any other beneficial effect that may be obtained.

Having thus described the basic concepts, it may be rather apparent to those skilled in the art after reading this detailed disclosure that the foregoing detailed disclosure is intended to be presented by way of example only and is not limiting. Although not explicitly stated here, those skilled in the art may make various modifications, improvements, and amendments to the present disclosure. These alterations, improvements, and amendments are intended to be suggested by this disclosure and are within the spirit and scope of the exemplary embodiments of the present disclosure.

Moreover, certain terminology has been used to describe embodiments of the present disclosure. For example, the terms “one embodiment,” “an embodiment,” and/or “some embodiments” mean that a particular feature, structure, or feature described in connection with the embodiment is included in at least one embodiment of the present disclosure. Therefore, it is emphasized and should be appreciated that two or more references to “an embodiment”, “one embodiment”, or “an alternative embodiment” in various portions of the present disclosure are not necessarily all referring to the same embodiment. In addition, some features, structures, or characteristics of one or more embodiments in the present disclosure may be properly combined.

Furthermore, the recited order of processing elements or sequences, or the use of numbers, letters, or other designations, therefore, is not intended to limit the claimed processes and methods to any order except as may be specified in the claims. Although the above disclosure discusses some embodiments of the invention currently considered useful by various examples, it should be understood that such details are for illustrative purposes only, and the additional claims are not limited to the disclosed embodiments. Instead, the claims are intended to cover all combinations of corrections and equivalents consistent with the substance and scope of the embodiments of the present disclosure. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. However, this disclosure does not mean that object of the present disclosure requires more features than the features mentioned in the claims. Rather, claimed subject matter may lie in less than all features of a single foregoing disclosed embodiment.

In some embodiments, the numbers expressing quantities or properties used to describe and claim certain embodiments of the present disclosure are to be understood as being modified in some instances by the term “about”, “approximate”, or “substantially”. For example, “about”, “approximate”, or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes. History application documents that are inconsistent or conflictive with the contents of the present disclosure are excluded, as well as documents (currently or subsequently appended to the present specification) limiting the broadest scope of the claims of the present disclosure. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the present disclosure disclosed herein are illustrative of the principles of the embodiments of the present disclosure. Other modifications that may be employed may be within the scope of the present disclosure. Thus, by way of example, but not of limitation, alternative configurations of the embodiments of the present disclosure may be utilized in accordance with the teachings herein. Accordingly, embodiments of the present disclosure are not limited to that precisely as shown and described.

What is claimed is:

1. A method for energy saving control of a medical device, comprising:
obtaining first historical operation data for the medical device of a user;
obtaining first predicted operation data by predicting, based on the first historical operation data, a user operation of the medical device, wherein the first predicted operation data includes a first predicted operation and a first predicted operation time corresponding to the first predicted operation;
determining, based on the first predicted operation data and current operation data, an energy-saving strategy for controlling the medical device, wherein the current operation data includes a current operation and a current time; and
controlling the medical device to implement the energy-saving strategy.

2. The method of claim 1, wherein the determining, based on the first predicted operation data and current operation data, an energy-saving strategy for controlling the medical device includes:
determining target predicted operation data of the user based on the first predicted operation data and the current operation data; and
determining the energy-saving strategy based on the target predicted operation data.

3. The method of claim 2, wherein the determining, based on the first predicted operation data and current operation data, an energy-saving strategy for controlling the medical device includes:
obtaining a predicted comparison result by comparing, according to a predetermined time threshold, the first predicted operation data and the current operation data; and
determining the target predicted operation data of the user and/or a target energy-saving strategy based on the predicted comparison result.

4. The method of claim 3, wherein the obtaining a predicted comparison result by comparing, according to a predetermined time threshold, the first predicted operation data and the current operation data includes:
obtaining the predicted comparison result by comparing a difference between the current time and the first predicted operation time with the predetermined time threshold.

5. The method of claim 4, wherein the predetermined time threshold includes a first predetermined value, and the determining, based on the first predicted operation data and current operation data, an energy-saving strategy for controlling the medical device includes:
in response to determining that the predicted comparison result is that the difference between the current time and the first predicted operation time is greater than the first predetermined value,
determining the first predicted operation time as a first target predicted operation time;
determining a first target predicted operation corresponding to the first target predicted operation time based on the first target predicted operation time; and
in response to determining that a first target component corresponding to the first target predicted operation is operating and the first target component is not a component corresponding to a target operation, determining the energy-saving strategy as controlling the first target component to stop operating, wherein
the target operation includes the current operation and the first predicted operation, the first predicted operation corresponding to the first predicted operation time having a difference with the current time less than or equal to the first predetermined value.

6. The method of claim 4, wherein the predetermined time threshold includes a second predetermined value, and the determining, based on the first predicted operation data and current operation data, an energy-saving strategy for controlling the medical device includes:
in response to determining that the predicted comparison result is that the difference between the current time and the first predicted operation time is less than the second predetermined value,
determining the first predicted operation time as a second target predicted operation time;
determining a second target predicted operation corresponding to the second target predicted operation time based on the second target predicted operation time; and
in response to determining that neither a component corresponding to the second target predicted operation nor a component corresponding to the current operation includes a second target component that is currently operating, determining the energy-saving strategy as controlling the second target component to stop operating.

7. The method of claim 4, wherein the predetermined time threshold includes a third predetermined value, and the determining, based on the first predicted operation data and current operation data, an energy-saving strategy for controlling the medical device includes:
in response to determining that the predicted comparison result is that the difference between the current time and the first predicted operation time is greater than the third predetermined value, determining the energy-saving strategy as controlling the medical device to enter a sleep mode if the current operation includes no operation.

8. The method of claim 1, wherein the obtaining a first predicted operation data by predicting, based on the first historical operation data, a user operation of the medical device includes:
obtaining the first predicted operation data by inputting the first historical operation data into a prediction model to predict a user operation of the medical device.

9. The method of claim 8, further comprising:
obtaining category information of the medical device; and
determining the prediction model corresponding to the category information based on the category information of the medical device.

10. The method of claim 9, wherein the category information of the medical device includes type information of the medical device, and the determining the prediction model corresponding to the category information based on the category information of the medical device includes:
determining the prediction model corresponding to the type information based on the type information of the medical device.

11. The method of claim 9, wherein the category information of the medical device includes log-in information of the user of the medical device, and the determining the prediction model corresponding to the category information based on the category information of the medical device includes:
determining the prediction model corresponding to the user based on the log-in information of the user.

12. The method of claim 8, further comprising:
obtaining first actual operation data corresponding to the first predicted operation data, wherein the first actual operation data includes a first actual operation and a first actual operation time corresponding to the first actual operation; and
determining whether to update the prediction model based on the first predicted operation data and the first actual operation data.

13. The method of claim 12, wherein the determining whether to update the prediction model based on the first predicted operation data and the first actual operation data includes:
determining reference predicted operation data and reference actual operation data, wherein the reference predicted operation data includes a reference predicted operation and a reference predicted operation time corresponding to the reference predicted operation, the reference actual operation data includes a reference actual operation and a reference actual operation time corresponding to the reference actual operation, the reference predicted operation includes any first predicted operation, and the reference actual operation is an operation that is in the first actual operation and the same as the reference predicted operation; and
determining whether to update the prediction model based on a difference between the reference predicted operation time and the reference actual operation time.

14. The method of claim 13, wherein the determining whether to update the prediction model based on a difference between the reference predicted operation time and the reference actual operation time includes:
comparing whether the difference between the reference predicted operating time and the reference actual operation time is greater than a fourth predetermined value;
updating the prediction model in response to determining that the difference between the reference predicted operation time and the reference actual operation time is greater than the fourth predetermined value; and
maintaining the prediction model unchanged in response to determining that the difference between the reference predicted operation time and the reference actual operation time is not greater than the fourth predetermined value.

15. The method of claim 13, wherein the determining whether to update the prediction model based on a difference between the reference predicted operation time and the reference actual operation time includes:
determining, for at least two reference predicted operations, whether differences between at least two reference predicted operation times and at least two reference actual operation times are greater than a fourth predetermined value;
in response to determining that the differences between the at least two reference predicted operation times and the at least two reference actual operation times are greater than the fourth predetermined value, updating the prediction model;
in response to determining that the differences between the at least two reference predicted operation times and the at least two reference actual operation times are not greater than the fourth predetermined value, maintaining the prediction model unchanged.

16. The method of claim 8, wherein the prediction model is obtained by training based on second historical operation data of the medical device of the user, wherein the second historical operation data includes a historical operation and a historical operation time corresponding to the historical operation.

17. The method of claim 16, wherein training the prediction model includes:
obtaining second predicted operation data by inputting sample data from the second historical operation data into the prediction model for predicting the user operation of the medical device, wherein the second predicted operation data includes a second predicted operation and a second predicted operation time corresponding to the second predicted operation;
determining a loss based on the second predicted operation data and the second actual operation data corresponding to the second predicted operation data in the second historical operation data, wherein the second actual operation data includes a second actual operation and a second actual operation time corresponding to the second actual operation, and the second actual operation time is later than the historical operation time in the sample data; and
obtaining a trained prediction model by adjusting a parameter of the prediction model according to the loss until a convergence condition is satisfied.

18. The method of claim 17, wherein the determining a loss based on the second predicted operation data and the second actual operation data corresponding to the second predicted operation data in the second historical operation data includes:
determining an operation error based on the second predicted operation and the second actual operation;
determining a time error based on the second predicted operation time and the second actual operation time; and
determining the loss based on the operation error and the time error.

19. A non-transitory computer readable medium, comprising executable instructions that, when executed by at least one processor, direct the at least one processor to perform a method for energy saving control of a medical device, the method comprising:
obtaining first historical operation data for the medical device of a user;
obtaining first predicted operation data by predicting, based on the first historical operation data, a user operation of the medical device, wherein the first predicted operation data includes a first predicted operation and a first predicted operation time corresponding to the first predicted operation;
determining, based on the first predicted operation data and current operation data, an energy-saving strategy for controlling the medical device, wherein the current operation data includes a current operation and a current time; and
controlling the medical device to implement the energy-saving strategy.

20. A device for energy saving control of a medical device, comprising at least one memory and at least one processor, wherein the at least one memory is configured to store computer instructions, and the at least one processor is configured to execute at least some of the computer instructions to realize a method for energy saving control, the method comprising:
obtaining first historical operation data for the medical device of a user;
obtaining first predicted operation data by predicting, based on the first historical operation data, a user operation of the medical device, wherein the first predicted operation data includes a first predicted operation and a first predicted operation time corresponding to the first predicted operation;
determining, based on the first predicted operation data and current operation data, an energy-saving strategy for controlling the medical device, wherein the current operation data includes a current operation and a current time; and
controlling the medical device to implement the energy-saving strategy.
